(19) United States
(12) Patent Application Publication
(10) Pub. No.: US 2007/0161806 A1
(43) Pub. Date: Jul. 12, 2007

(54) Catalyst for the carbonylation of oxiraines

(75) Inventors: Peter Preishuber-Pflugl, Ludwigshafen (DE); Ferenc Molnar, Speyer (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/586,826

(22) PCT Filed: Jan. 20, 2005
(86) PCT No.: PCT/EP05/00534

§ 371 Date: Aug. 8, 2006

(30) Foreign Application Priority Data
Jan. 20, 2004 (DE) 10 2004 002 875.3

(51) Int. Cl. C07D 305/12

(52) U.S. Cl. 549/263; 549/328

(57) ABSTRACT

Lactones are prepared by the catalytic carbonylation of oxiranes using a catalyst system comprising a) at least one carbonylation catalyst A comprising uncharged or anionic transition metal complexes of metals of groups 5 to 11 of the Periodic Table, b) at least one metal compound B of the formula (I)
MXₓRₙ₋ₓ (I) where M is an element of group 2, 3, 4, 12, 13, R is hydrogen or a hydrocarbon radical which may be substituted on the carbon atoms other than on the carbon atom bound to M, X is an anion, n is a number corresponding to the valence of M, x is in the range from 0 to n, and c) at least one organic, chiral compound C which has fewer than four coordination sites.

The present invention relates to the preparation of enantiomerically enriched lactones by catalytic carbonylation of oxiranes in the presence of a catalyst system, to a corresponding catalyst system, and to a process for its preparation and its use.

In particular, the invention relates to the preparation of enantiomerically enriched four-membered ring lactone mixtures from racemic epoxides by catalytic carbonylation. The catalysis is achieved by means of a catalyst system comprising three components. The optically enriched mixture of R- and S-lactones can be converted into a biodegradable polyester.

Lactones are valuable compounds for preparing biodegradable polyesters, as described, for example, in EP-A 0 688 806. These polyesters are widely used, for example as polyol in polyurethane production or as material of construction.

The properties of such β-alkylhydroxyalkanoate polyesters depend greatly on the stereoregularity. Thus, for example, atactic polyhydroxybutyrate (PHB) is a viscous oil, while isotactic PHB is a solid which can be used as a material of construction. Isotactic PHB can be obtained from enantiomerically pure butyrolactone. Enantiomerically pure butyrolactone can be prepared either by means of a complicated organic synthesis using protective groups or by a biochemical route. However, the synthetic route to these compounds is, particularly for large-scale industrial applications, very complex and costly. In addition, the purification is also complicated.

Furthermore, the processing of pure isotactic PHB by means of injection molding is problematical, since the decomposition temperature is very close to the melting point. In addition, pure isotactic PHB is brittle. A more readily processable and tougher PHB is not purely isotactic, but instead contains proportions of atactic structural elements. Such a PHB can be obtained from mixtures of R- and S-butyrolactones by polymerization. It is therefore desirable to prepare lactone mixtures in which one enantiomer is present in an excess over the other enantiomer. The preparation of lactones by catalytic carbonylation of simple and substituted oxiranes is known per se. The products are often not the desired lactones, or the reaction conditions or the starting materials do not permit efficient preparation or isolation of lactones. The compounds can frequently be obtained only by means of complicated and costly syntheses.

JP-A-09 169 753 describes the carbonylation of epoxides to lactones over Co₂(CO)₈ as catalyst in a flow-through reactor. The conversions are only 30%. This means that a separation and recirculation facility is required to achieve high yields and purity of the lactone.

GB-A-1,020,575 relates to a process for preparing polymers of β-lactones. Carbon monoxide and a 1,2-epoxide are reacted to form a β-lactone as intermediate. Octacarbonyldicobalt is used as catalyst in this reaction. In addition, a promoter selected from among metal halides such as potassium iodide and quaternary ammonium halides such as tetraethylammonium bromide can be added. However, the yields of lactone are less than 10%, and the main fractions of the products are polyhydroxypropionic esters. In addition, the reaction is carried out in a complicated manner with a plurality of pressure stages.

EP-B-0 577 206 relates to the carbonylation of epoxides over a catalyst system comprising a cobalt source and a hydroxy-substituted pyridine compound, in particular 3-hydroxypyridine or 4-hydroxypyridine. The carbonylation is preferably carried out in the presence of a hydroxy compound such as water or alcohols. The activities of the catalysts used are relatively low, and isolation of the lactones is not described. It was also observed that a change in the reaction mixture occurred after the carbonylation had ended. Polymerization of the lactone takes place within 24 hours. This indicates that the lactone is not unreactive in the reaction mixture. It is also known that lactones can be polymerized in the presence of pyridines.

Chemistry Letters 1980, pages 1549 to 1552, relates to the reaction of epoxides with carbon monoxide over a rhodium complex as catalyst. The yields are not more than 70%.

J. Org. Chem. 2001, 66, pages 5424 to 5426, describes the synthesis of β-lactones by carbonylation of epoxides over cobalt and Lewis acid catalysts. As catalyst, use is made of a system comprising PPNCo(CO)₄ and BF₃.Et₂O. The yields range from 7 to 86%. However, the reaction time is from 7 to 24 hours and the use of large amounts of catalyst is necessary.

J. Am. Chem. Soc. 124, No. 7, 2002, pages 1174 to 1175, describes the preparation of β-lactones by carbonylation of epoxides. The catalyst used is a mixture of aluminum salts and a tetracarbonylcobaltate. A mixture of lactones having an excess of one enantiomer is not obtained.

J. Org. Chem. 1999, 64, pages 2164 to 2165, describes the preparation of chiral epoxides and hydroxy alcohols from racemic epoxides using a chiral Co(salen)(N,N-bis-[3,5-di-t-butylsalicylidene]-1,2-diaminocyclohexane). In a subsequent step, the epoxides are reacted with a mixture of Co/H₂ and propanols in the presence of octacarbonyldicobalt to give chiral acetals. Two steps are thus necessary for the preparation of carbonylated compounds based on epoxides. This is complicated, does not give a lactone and, in addition, half of the racemic oxirane is lost.

WO 03/050154 relates to the use of compounds comprising cationic Lewis acids and anionic metal carbonyl compounds for the carbonylation of epoxides. However, the synthetic route to these compounds is complicated and relatively unsuitable for industrial use. An enantiomerically enriched mixture of lactones was not obtained when starting from racemic epoxides.

M. Allmendiger, Thesis, Univ. Ulm, 2003, pages 109 to 115, describes the preparation of enantiomerically enriched four-membered ring lactone mixtures. A combination of a transition metal complex and a chiral Lewis acid is used for the asymmetric carbonylation. A chromium-salen complex is used as preferred chiral Lewis acid. An enantiomeric excess of 14% could be achieved therewith. It is also stated that an aluminum compound having the same ligand does not achieve any enantio selectivity.

It is an object of the present invention to provide an uncomplicated and efficient process for the preparation and isolation of optically enriched β-lactones.

We have found that this object is achieved by a process for preparing lactones by catalytic carbonylation of oxiranes using a catalyst system comprising
a) at least one carbonylation catalyst A comprising uncharged or anionic transition metal complexes of metals of groups 5 to 11 of the Periodic Table of the Elements,
b) at least one metal compound B of the formula (I)
   MXₓRₙ₋ₓ (I)
where
   M is an element of group 2, 3, 4, 12, 13,
   R is hydrogen or a hydrocarbon radical which may be substituted on the carbon atoms other than on the carbon atom bound to M,
   X is an anion,
   n is a number corresponding to the valence of M,
   x is in the range from 0 to n, and
c) at least one organic, chiral compound C which has fewer than four coordination sites.

According to the present invention, it has been found that a catalyst system comprising three components, viz. a carbonylation catalyst A, a metal compound B and a chiral compound C, leads to optically enriched lactones in the carbonylation of oxiranes.

In addition, it has been found that the combination of the carbonylation catalyst A with the metal compounds B and the chiral compounds C allows efficient catalysis of the carbonylation of oxiranes to lactones under mild conditions.

The lactones obtained can advantageously be used for the preparation of biodegradable polyesters which can be used as polyol in polyurethane production or as materials of construction.

In the catalyst system used according to the present invention, the components A, B and C are preferably present in a ratio of from 1:0.1:0.1 to 1:100:100, particularly preferably in a ratio of from 1:1:1 to 1:10:100, very particularly preferably from 1:2:2 to 1:10:20.

Carbonylation catalysts A can in principle be any complexes based on metals of groups 5 to 11 of the Periodic Table of the Elements. Examples of suitable metals are vanadium, ruthenium, chromium, molybdenum, tungsten, manganese, rhenium, iron, osmium, cobalt, iridium, rhodium and nickel. Such complexes can also be generated in situ, cf. EP-A 0 577 206. Particular preference is given to Re, Co, Ru, Rh, Fe, Ni, Mn, Mo, W or mixtures thereof, in particular Co.

In the uncharged transition metal complex (A), the ligands are generally present as uncharged ligands. The number of ligands depends on the respective metal and is determined by the coordinative saturation of the transition metal in the ground state. Suitable uncharged ligands are, for example, carbon monoxide, nitro, nitroso, carbonate, ether, sulfoxide, amide, nitrile, phosphite or phosphine ligands. These ligands are generally coordinated to the transition metal via a free electron pair. Preference is given to using carbon monoxide as ligand. It is also possible for different ligands to be present together in a transition metal compound (A), as in Co₂(CO)₆(PMe₂Ph)₂. Preferred transition metal complexes (A) are Co₂(CO)₈, Ru₃(CO)₁₂, Rh₄(CO)₁₂, Rh₆(CO)₁₆, Co₄(CO)₁₂, Fe₂(CO)₁₀, Fe₂(CO)₉, Ni(CO)₄, Mn₂(CO)₁₀, Mo(CO)₆ and W(CO)₆ or mixtures thereof. Particular preference is given to Ru₃(CO)₁₂, Co₄(CO)₁₂, Co(CO)₃(NO), Ni(CO)₄ and Mn₂(CO)₁₀, in particular Co₂(CO)₈.

It is also possible to use mixtures of various uncharged complexes.

The preparation of the uncharged transition metal complexes A is generally known to those skilled in the art and is described, for example, in F. G. Stone, E. W. Abel and G. Wilkinson, “Comprehensive Organometallic Chemistry—The Synthesis, Reactions and Structures of Organometallic Compounds”, Pergamon Press, Oxford, 1982, for example in Vol. 5. Furthermore, such complexes are also commercially available.

For the purposes of the present invention, transition metal complexes (A) include compounds in which at least one central metal or a ligand unit bears a formal negative charge. Suitable anionic transition metal compounds (A) have a central metal from groups 5 to 11, preferably from groups 8 to 10, of the Periodic Table of the Elements. Possible metals are, for example, cobalt, iron, rhodium and ruthenium. Particular preference is given to using transition metal complexes (A) based on the metals cobalt, ruthenium and rhodium. It is possible to use mononuclear or multinuclear complexes A.

In the anionic transition metal complex A, the ligands are usually also present as uncharged ligands. The number of ligands depends on the respective metal and is determined by the coordinative saturation of the transition metal in the ground state. Examples of suitable uncharged ligands are carbon monoxide, nitro, nitroso, carbonate, ether, sulfoxide, amide, nitrile, phosphite or phosphine ligands. These ligands are generally coordinated to the transition metal via a free electron pair. Preference is given to using carbon monoxide as ligand. It is also possible for different ligands to be present together in an anionic transition metal compound A, for example as in [P(Ph)₃]Co(−1)(CO)₃, [P(Me₂Ph)]Co(−1)(CO)₃, Co(−1)(CO)₃(CNPh). These compounds, too, can be generated in situ.

Suitable anionic transition metal complexes A have, for example, the formula (II):
(M_{α}⁽ⁿ⁺⁾)ₘ[M_{β}(L)₄]_{I} (II),
where the variables and indices have the following meanings:
M_{β} is a transition metal of groups 8 to 10 of the Periodic Table of the Elements, in particular cobalt or rhodium, bearing the formal charge −1,
L is PR₃, P(OR)₃, NR₃, SR₂, OR₂, CO, R—CN, R—NO₂, (RO)(R′O)C═O, (R)(R′)C═O, (R)C═O(OR′), in particular CO,
M_{α} is a metal of group 1 or 2 of the Periodic Table of the Elements, Zn or Hg, in particular Na, K, Cs, Mg, Ca, Zn and Hg, bis(triarylphosphine)iminium, trityl or T(R)₄ where
T is N, P or As, in particular N,
R, R′ are each, independently of one another, H, alkyl, aryl, alkaryl or aralkyl,
n, m are each 1 or 2 and
I is n×m.

Possible radicals R, R′ are, for example, hydrogen, straight-chain or branched C₁-C₁₀-alkyl such as methyl, ethyl, n- or i-propyl, n-, i- or t-butyl or n- or i-pentyl, C₆-C₁₄-aryl such as phenyl or naphthyl or alkylaryl having from 1 to 10 carbon atoms in the alkyl part and from 6 to 14 carbon atoms in the aryl part, e.g. benzyl. Suitable aromatic radicals also include heterocycles and may be, for example 5- or 6-membered monocycles such as pyridyl and phenyl, and also fused systems such as anthracene.

Among the nonmetallic cations M, preference is given to tetraphenyl-, tetramethyl-, tetraethyl- and tetra-n-butyl-ammonium, -phosphonium and -arsenium and also bis(triarylphosphine)iminium. Particularly useful aryl radicals in the bis(triarylphosphine)iminium cation are phenyl and naphthyl, with bis(triphenylphosphine)iminium being preferred.

Possible metallic cations M_{α} include alkali metal and alkaline earth metal cations. Preference is given to using lithium, sodium, potassium and cesium.

Use is advantageously made of anionic transition metal complexes A selected from the group consisting of Li[Co(CO)₄], Na[Co(CO)₄], K[Co(CO)₄], Cs[Co(CO)₄], (R₄N)[Co(CO)₄], (R₄P)[Co(CO)₄], (R₄As)[Co(CO)₄], (PPN)[Co(CO)₄], Li[Rh(CO)₄], Na[Rh(CO)₄], K[Rh(CO)₄], Cs[Rh(CO)₄], (R₄N)[Rh(CO)₄], (R₄P)[Rh(CO)₄], (R₄As)[Rh(CO)₄], (PPN)[Rh(CO)₄], Li[Ir(CO)₄], Na[Ir(CO)₄], K[Ir(CO)₄], Cs[Ir(CO)₄], (R₄N)[Ir(CO)₄], (RrP)[Ir(CO)₄], (R₄As)[Ir(CO)₄], (PPN)[Ir(CO)₄], Li₂[Fe(CO)₄], Na₂[Fe(CO)₄], K₂[Fe(CO)₄], Cs₂[Fe(CO)₄], (R₄N)₂[Fe(CO)₄], (R₄P)₂[Fe(CO)₄], (R₄As)₂[Fe(CO)₄], (PPN)₂[Fe(CO)₄], (PPN)[HFe(CO)₄] and (PPN)₂[Fe₂(CO)₈, where R is methyl, ethyl, n- or i-propyl, n-, i- or t-butyl, phenyl or benzyl.

Among anionic complexes A containing cobalt in the oxidation state −1, particular preference is given to tetraphenylphosphonium tetracarbonylcobaltate, tetraphenylarsenium tetracarbonylcobaltate, tetraphenylammonium tetracarbonylcobaltate, tetraethylphosphonium tetracarbonylcobaltate, tetraethylarsenium tetracarbonylcobaltate and tetraethylammonium tetracarbonylcobaltate and also sodium tetracarbonylcobaltate.

It is of course also possible to use mixtures of anionic and/or uncharged transition metal complexes A.

The preparation of anionic transition metal complexes is generally known to those skilled in the art. Suitable preparative methods may be found, for example, in F. G. Stone, E. W. Abel and G. Wilkinson, “Comprehensive Organometallic Chemistry—The Synthesis, Reactions and Structures of Organometallic Compounds”, Pergamon, Oxford, 1982 and F. G. Stone, E. W. Abel and G. Wilkinson, “Comprehensive Organometallic Chemistry II—A Review of the Literature 1982-1994”, Pergamon Press, Oxford for example in Vol. 8. Furthermore, such complexes are also commercially available.

The molar ratio of anionic complex or uncharged complex (A) in the reaction mixture is usually in the range from 0.01 to 100 mol %, preferably from 0.1 to 50 mol %, particularly preferably from 0.2 to 10 mol %, based on the amount of oxirane used.

The component A is selected so that a carbonyl compound is present under the reaction conditions.

In the metal compound B of the formula (I)
MXₓRₙ₋ₓ (I)

M is preferably an element of group 2, 3, 4, 12 or 13. M is particularly preferably a metal selected from the group consisting of Al, Mg, Zn, Ti, Zr and Sn. Very particular preference is given to M being Al.

R is preferably hydrogen or C₁₋₃₂-alkyl, C₂₋₂₀-alkenyl, C₃₋₂₀-cycloalkyl, C₆₋₁₈-aryl, C₇₋₂₀-aralkyl or C₇₋₂₀-alkaryl, where substituents may be present on the carbon atoms other than the carbon atom bound to M. R is preferably hydrogen or a monoanionic hydrocarbyl group, for example C₁₋₃₂-alkyl such as methyl, ethyl, i- or n-propyl, i-, n- or t-butyl, n-pentyl or n-hexyl, C₂₋₂₀-alkenyl such as propenyl or butenyl, C₃₋₂₀-cycloalkyl such as cyclopropyl, cyclobutyl, cyclopentyl, cyclopentadienyl or cyclohexyl, C₆₋₁₈-aryl such as phenyl or naphthyl, or C₇₋₂₀-arylalkyl, e.g. benzyl (the preferred hydrocarbyl group is alkyl, and particularly preferred hydrocarbyl groups are methyl and ethyl),

X is preferably an anion such as Cl, Br, I, sulfonate, oxide, C₁₋₃₂-alkoxide, amide, preferred anions are Cl, Br, I and alkoxide, particularly preferably chloride or C₁₋₁₂-alkoxide,

where n corresponds to the oxidation state OS or the valence of the metal, x is less than or equal to n and is not negative (for each oxide ligand, x=x+1).

The metal compound B is preferably AlXₓR₃₋ₓ, where x is from 0 to 3 and R is C₁₋₆-alkyl. The numbers n and x can be integers or fractions. Fractions can occur in the case of a mixture of such compounds.

The component B is particularly preferably an aluminum alkyl or alkylaluminum halide compound.

The component A or B may, if appropriate, also have an uncharged donor L in the coordination sphere. The donor L is generally an uncharged compound containing oxygen, nitrogen or phosphorus atoms, for example ethers, carbonates, ketones, sulfoxides, amines, amides, phosphanes, nitro or nitrile, etc., functions. The donor L can also be an olefin or aromatic.

The component C is an organic, chiral compound having less than 4 coordination sites.

The component C is preferably a metal-free organic, chiral compound.

The component C is preferably a low molecular weight organic, chiral compound having at least five carbon atoms.

The component C is preferably an organic, chiral compound whose melting point or boiling point is >20° C.

The component C is preferably an organic, chiral compound containing heteroatoms such as N, O, S, P, halogens.

Preference is given to using chiral compounds selected from the group consisting of oxazolines, imines, amines, alcohols, carboxylic acids and amino acids as component C.

The component C is particularly preferably a bisoxazoline compound or a chiral alcohol, or it comprises a chiral alcohol.

Very particular preference is given to using 2,2′-methylenebis[(4R,5S)-4,5-diphenyl-2-oxazoline, 2,2′-methylenebis[(R)-4-phenyl-2-oxazoline, (+)-(4S)-phenyl-alpha-[(4S)-phenyloxazolidin-2-ylidene]-2-oxazoline-2-acetonitrile, menthol, (R)-(+)-1,1′-bi-2-naphthol.

The compounds which can be used according to the present invention as component C are commercially available or can be prepared by methods known to those skilled in the art.

Of course, it is also possible to employ a plurality of different components A and/or B and/or C as a catalyst system.

Particular preference is given to a combination selected from the group consisting of:
sodium tetracarbonylcobaltate with dimethylaluminum chloride and 2,2′-methylenebis[(4R,5S)-4,5-diphenyl-2-oxazoline,
sodium tetracarbonylcobaltate with trimethylaluminum and 2,2′-methylenebis[(4R,5S)-4,5-diphenyl-2-oxazoline,
sodium tetracarbonylcobaltate with monomethylaluminum dichloride and 2,2′-methylenebis[(4R,5S)-4,5-diphenyl-2-oxazoline,
octacarbonyldicobaltate with trimethylaluminum and 2,2′-methylenebis[(4R,5S)-4,5-diphenyl-2-oxazoline,
sodium tetracarbonylcobaltate with dimethylaluminum chloride and (+)-(4S)-phenyl-alpha-[(4S)-phenyloxazolidin-2-ylidene]-2-oxazoline-2-acetonitrile,
sodium tetracarbonylcobaltate with monomethylaluminum dichloride and (+)-(4S)-phenyl-alpha-[(4S)-phenyloxazolidin-2-ylidene]-2-oxazoline-2-acetonitrile,
octacarbonyidicobaltate with trimethylaluminum and (+)-(4S)-phenyl-alpha-[(4S)-phenyloxazolidin-2-ylidene]-2-oxazoline-2-acetonitrile,
sodium tetracarbonylcobaltate with dimethylaluminum chloride and menthol,
sodium tetracarbonylcobaltate with trimethylaluminum and menthol, and
sodium tetracarbonylcobaltate with dimethylaluminum chloride and (R)-(+)-1,1′-bi-2-naphthol.

The carbonylation is generally carried out under superatmospheric pressure and at elevated temperature. However, product formation is also observed at a carbon monoxide pressure of one atmosphere. The pressure is generally generated by means of CO gas. This pressure can in particular cases also be generated partially by an inert medium such as argon, nitrogen. The pressures are in the range from 1 to 250 bar, preferably from 10 to 100 bar, particularly preferably from 20 to 80 bar. The reaction can generally be carried out at temperatures of from −10 to 200° C. The preferred temperature is from 20 to 150° C., particularly preferably from 40 to 110° C.

The carbonylation of epoxides can be carried out either batchwise or in a continuous process. It can be carried out either in the gas phase or in an inert reaction medium. This medium is generally a liquid. Such liquids can be customary solvents as ether, diglyme, triglyme, tetraglyme, tetrahydrofuran, dimethoxyethane, hydrocarbons such as hexane, octane, Isopar, benzene, toluene, xylene, decalin; chlorinated hydrocarbons such as dichloromethane, dichloroethane, dichlorobenzene or polar solvents such as DMF, DMSO, esters, nitriles, nitro compounds, ketones or ionic liquids. Preferred solvents are DME, diglyme, dichloromethane. The oxirane can also be used as reaction medium.

To activate the catalyst system further, it is possible to add donor ligands such as phosphanes or nitriles. Applying the catalyst components (e.g. cobalt compound, alkyl compound and chiral compound) to a particulate support material, e.g. silica or aluminum oxide, also makes it possible to carry out the reaction in a solvent-free fashion as a gas-phase carbonylation.

Suitable oxirane compounds are ethylene oxide and substituted epoxides. These are usually compounds having the formula (III):

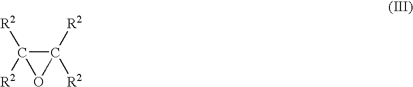

In this formula, the radicals R² are each, independently of one another, hydrogen, halogen, a nitro group —NO₂, a cyano group —CN, an ester group —COOR³ or a hydrocarbon group having from 1 to 32 carbon atoms which may be substituted. The radicals R² in a compound of the formula (III) can all be the same, some of them can be the same or they can be four different radicals. R³ can be C₁₋₁₂-alkyl, aryl.

Preference is given to using geminally substituted epoxides, particularly preferably epoxides which are substituted only in the 1 position.

Suitable hydrocarbon groups are, for example, C₁₋₃₂-alkyl such as methyl, ethyl, i- or n-propyl, i-, n- or t-butyl, n-pentyl or n-hexyl, C₂₋₂₀-alkenyl such as propenyl or butenyl, C₃₋₂₀-cycloalkyl such as cyclopropyl, cyclobutyl, cyclopentyl or cyclohexyl, C₆₋₁₈-aryl such as phenyl or naphthyl, and C₇₋₂₀-arylalkyl, e.g. benzyl. It is also possible for two radicals R² located on different carbon atoms of the epoxy group to be joined to one another and thus form a C₃₋₂₀-cycloalkylene group.

Possible substituents by which the C₁₋₃₂-hydrocarbon group and also R above can be substituted are, in particular, the following groups: halogen, cyano, nitro, thioalkyl, tertamino, alkoxy, aryloxy, arylalkyloxy, carbonyidioxyalkyl, carbonyldioxyaryl, carbonyldioxyarylalkyl, alkoxycarbonyl, aryloxycarbonyl, arylalkyloxycarbonyl, alkylcarbonyl, arylcarbonyl, arylalkylcarbonyl, alkylsulfinyl, arylsulfinyl, arylalkylsulfinyl, alkylsulfonyl, arylsulfonyl and arylalkylsulfonyl.

As oxirane compound, preference is given to using ethylene oxide, propylene oxide, butylene oxide (1-butene oxide, BuO), cyclopentene oxide, cyclohexene oxide (CHO), cycloheptene oxide, 2,3-epoxypropyl phenyl ether, epichlorohydrin, epibromohydrin, i-butene oxide (IBO), styrene oxide or acrylic oxides. Particular preference is given to using ethylene oxide (EO), propylene oxide (PO), butylene oxide or i-butene oxide, very particularly preferably ethylene oxide or propylene oxide or a mixture thereof.

The oxirane compounds to be used in the process of the present invention can be obtained, for example, by epoxidations of terminal olefins in a manner known to those skilled in the art.

Possible compounds having a terminal double bond are in principle all olefins of this class of compounds, e.g. propene, 1-butene, 1-pentene, 1-hexene, 1-heptene or 1-octene.

In general, the components A, B and C are firstly introduced into the reaction vessel either individually, simultaneously or in premixed form, if appropriate with cooling. It is also possible, if desired, for the oxirane compound to be mixed into the solution/suspension of the catalyst components before the latter is transferred to the reaction vessel. Furthermore, the oxirane compound can also be introduced directly into the reaction vessel. The carbonylation is preferably carried out under inert conditions, i.e. in the absence of moisture and air.

Termination, separation and purification of the lactones can be carried out by generally known methods. For example, the lactone can be isolated in a simple fashion by distillation or crystallization.

The process of the present invention enables enantiomerically enriched 3-hydroxypropiolactones to be obtained from the corresponding racemates of oxirane compounds. Use of oxirane compounds in optically enriched form results in lactones whose optical purity is higher than the optical purity of the oxirane serving as starting material. Use of the lactones prepared in this way make it possible to produce the thermoplastic property profile of the biodegradable polymers whose properties can be set very simply and specifically for desired applications.

The advantages of the invention are also apparent in the simple procedure and in the high activity and productivity of the carbonylation catalysis and of the commercially available catalyst components.

The invention also provides a process for preparing the catalyst system used according to the present invention by mixing the components A, B and C.

Furthermore, the invention relates to the catalyst itself and also the use of the catalyst in carbonylation reactions.

### EXAMPLES

To carry out the carbonylation, the appropriate cobalt compound A, the metal compound B and the organic, chiral compound C were placed in a Parr steel autoclave and dissolved in giglyme. The oxirane was subsequently added. A carbon monoxide pressure of 60 bar was applied and the mixture was stirred at 80° C. for 6 hours. The enantiomeric excesses determined by gas chromatography on a chiral stationary phase.

Results

| | | | | Δ0 | Conversion | ee [%, |
|---|---|---|---|---|---|---|
| No. | A [mmol] | B [mmol] | C [mmol] | [bar] | into BL [%] | conf] |
| 1 | 0.02 mmol of | 0.06 mmol of | 0.06 mmol of 2,2′-methylenebis[(4R,5S)-4,5- | 5 | 6 | 1.6, S |
| | NaCoCO₄ | Me₂AlCl | diphenyl-2-oxazoline | | | |
| 2 | 0.03 mmol of | 0.09 mmol of | 0.09 mmol of 2,2′-methylenebis[(4R,5S)-4,5- | 5 | 4 | 1.0 S |
| | NaCoCO₄ | Me₂AlCl | diphenyl-2-oxazoline | | | |
| 3 | 0.03 mmol of | 0.09 mmol of | 0.09 mmol of 2,2′-methylenebis[(4R,5S)-4,5- | 7 | 6 | 4.4, R |
| | NaCoCO₄ | Me₃Al | diphenyl-2-oxazoline | | | |
| 4 | 0.03 mmol of | 0.09 mmol of | 0.09 mmol of 2,2′-methylenebis[(4R,5S)-4,5- | 2 | 3 | 1.4, S |
| | NaCoCO₄ | MeAlCl₂ | diphenyl-2-oxazoline | | | |
| 5 | 0.03 mmol of | 0.09 mmol of | 0.09 mmol of 2,2′-methylenebis[(4R,5S)-4,5- | 5 | 1 | 10.0, R |
| | Co₂CO₈ | Me₃Al | diphenyl-2-oxazoline | | | |
| 6 | 0.02 mmol of | 0.06 mmol of | 0.06 mmol of (+)-(4S)-phenyl-alpha-[(4S)- | 4 | 4 | 1.6, S |
| | NaCoCO₄ | Me₂AlCl | phenyloxazolidin-2-ylidene]-2-oxazoline-2- | | | |
| | | | acetonitnie | | | |
| 7 | 0.03 mmol of | 0.09 mmol of | 0.09 mmol of (+)-(4S)-phenyl-alpha-[(4S)- | 11 | 7 | 11.0, S |
| | NaCoCO₄ | MeAlCl₂ | phenyloxazolidin-2-ylidene]-2-oxazoline-2- | | | |
| | | | acetonitrile | | | |
| 8 | 0.03 mmol of | 0.09 mmol of | 0.09 mmol of (+)-(4S)-phenyl-alpha-[(4S)- | 33 | 25 | 2.8, 5 |
| | Co₂CO₈ | Me₃Al | phenyloxazolidin-2-ylidene]-2-oxazoline-2- | | | |
| | | | acetonitrile | | | |
| 9 | 0.03 mmol of | 0.09 mmol of | 0.09 mmol of menthol | 14 | 11 | 1.0, S |
| | NaCoCO₄ | Me₂AlCl | | | | |
| 10 | 0.03 mmol of | 0.09 mmol of | 0.09 mmol of menthol | 20 | 18 | 1.4, S |
| | NaCoCO₄ | Me₃Al | | | | |
| 11 | 0.03 mmol of | 0.09 mmol of | 0.18 mmol of menthol | 17 | 13 | 1.4, S |
| | NaCoCO₄ | Me₂AlCl | | | | |
| 12 | 0.02 mmol of | 0.06 mmol of | 0.18 mmol of menthol | 3 | 5 | 6.8, S |
| | NaCoCO₄ | Me₃Al | | | | |
| 13 | 0.02 mmol of | 0.06 mmol of | 0.06 mmol of (R)-(+)-1,1′-bi-2-naphthol | 4 | 4 | 1.8, S |
| | NaCoCO₄ | Me₂AlCl | | | | |

1-10. (canceled)

11. A process for preparing lactones by catalytic carbonylation of oxiranes using a catalyst system comprising
a) at least one carbonylation catalyst A comprising uncharged or anionic transition metal complexes of metals of groups 5 to 11 of the Periodic Table of the Elements,
b) at least one metal compound B of the formula (I)
MXₓRₙ₋ₓ (I)
where
M is an element of group 2, 3, 4, 12, 13,
R is hydrogen or a hydrocarbon radical which may be substituted on the carbon atoms other than on the carbon atom bound to M,
X is an anion,
n is a number corresponding to the valence of M,
x is in the range from 0 to n, and
c) at least one organic, chiral compound C that is a bisoxazoline compound and/or comprises at least one chiral alcohol.

12. The process as claimed in claim 11, wherein enantiomerically enriched lactones are obtained in the process.

13. The process as claimed in claim 11, wherein the component A is selected so that a cobalt carbonyl compound is present under the reaction conditions.

14. The process as claimed in claim 11, wherein M in the formula (I) is Al, Mg, Zn, Ti, Zr or Sn.

15. The process as claimed in claim 11, wherein, in the formula (I), R is hydrogen or C₁₋₃₂-alkyl, C₂₋₂₀-alkenyl, C₃₋₂₀-cycloalkyl, C₆₋₁₈-aryl, C₇₋₂₀-aralkyl or C₇₋₂₀-alkaryl, where substituents may be present on the carbon atoms other than the carbon atom bound to M,
and/or X is Cl, Br, I, sulfonate, oxide, C₁₋₃₂-alkoxide or amide.

16. The process as claimed in claim 11, wherein the component B is AlClₓR₃₋ₓ, where x is from 0 to 3 and R is C₁₋₆-alkyl.

17. The process for preparing a catalyst system by mixing the components A, B and C as set forth in claim 11 in any order.

18. A catalyst system comprising the components A, B, C as defined in claim 11.

19. A method of using a catalyst system as claimed in claim 18 in carbonylation reactions.
